(19) United States
(12) Patent Application Publication
(10) Pub. No.: US 2011/0066137 A1
(43) Pub. Date: Mar. 17, 2011

(54) APPLICATOR FOR APPLYING POWDER FORMULATIONS AND USES THEREOF

(75) Inventors: Thomas Parks, San Mateo, CA (US); Troy Just, Lincoln, NE (US)

(73) Assignee: OSEL, Inc., Santa Clara, CA (US)

(21) Appl. No.: 12/809,043

(22) PCT Filed: Dec. 17, 2008
(86) PCT No.: PCT/US08/87288

§ 371 Date: Oct. 20, 2010

Related U.S. Application Data
(62) Division of application No. 61/014,673, filed on Dec. 18, 2007.

(51) Int. Cl. A61M 31/00

(52) U.S. Cl. 604/515; 604/58

(57) ABSTRACT

The present invention relates to applicators for applying powders to the vaginal mucosa.

### CROSS-REFERENCES TO RELATED APPLICATIONS

This application claims priority to U.S. Provisional Application No. 61/014,673, filed Dec. 18, 2007, the contents of which are herein incorporated by reference.

### BACKGROUND OF THE INVENTION

The mucosal membranes of all humans are naturally colonized by bacteria. Recent scientific evidence has documented the fact that these bacteria interact closely with cells and tissues of the body to regulate natural biological processes. It has become increasingly evident that this mucosal microflora also contributes substantially to numerous diseases affecting cells and tissues of humans.

Generally, domination of the microflora within the vagina by lactobacilli is associated with good vaginal health. Exogenous strains of lactobacilli have been administered for many years as “probiotics” for the purpose of maintaining a healthy microflora and preventing infection. These “healthy bacteria” compete with pathogenic organisms, such as bacteria, viruses and fungi to limit the development and progression of pathogen associated diseases. Nevertheless, this microflora is a fragile and dynamic environment with the natural turnover and disruption of the healthy microflora being associated with the establishment of opportunistic infections. Consequently, approaches to maintain, or even enhance, the integrity and natural properties of the microflora, as a means of preventing or treating disease, are useful.

The first natural line of defense against disease transmission for women during heterosexual contact is the mucosal barrier in the vagina. In healthy American and European women of childbearing age, Lactobacilli are most commonly the predominant bacteria colonizing the vaginal mucosa (Antonio et al., J. Infect. Dis. 180:1950-1956 (1999); Vasquez et al., J. Clin. Microbiol. 40:2746-2749 (2002)). These lactobacilli secrete lactic acid as a byproduct of metabolism, serving to acidify the vaginal mucosa and to keep the numbers of competing pathogenic microorganisms relatively low. Additionally, the predominance of hydrogen peroxide-producing strains of Lactobacillus in the vaginal microflora of healthy women has been associated with a lowered incidence of bacterial vaginosis and urinary tract infections. Lactobacillus crispatus and Lactosbacillus jensenii are the predominate hydrogen peroxide-producing species in the normal vaginal microflora. Further, predominance of lactobacilli has been associated as well with a lowered incidence of HIV acquisition, perhaps correlating with the observed antagonistic effects of metabolic byproducts of lactobacilli (lactic acid and hydrogen peroxide), on the infectivity of the HIV virus in vitro. Therefore, beneficial lactobacilli associated with the vaginal mucosa can be considered to provide a protective “biofilm”. See generally, Falagas et al., “Probiotics for prevention of recurrent urinary tract infections in women: a review of the evidence from microbiological and clinical studies,” Drugs, 66(9):1253-61 (2006).

Various methods for administering beneficial bacteria and other substances to the vaginal mucosa are known. For example, dried lactobacilli have been administered in vaginal tablets, in capsules, or in vaginal suppositories. Nonetheless, each of these methods has its drawbacks, and additional methods would be useful to provide more choices to females wishing to administer therapeutic bacteria or other agents to the vaginal mucosa. A further advantage of the present invention, over those previously used, is the delivery of a bacteria powder formulation directly into the vagina facilitates optimal bacterial rehydration and colonization of the vaginal mucosa. The present invention addresses these and other needs.

### BRIEF SUMMARY OF THE INVENTION

In a first group of embodiments, the invention provides applicators for applying powders to the vagina. The applicators comprise a) a first member capable of housing a powder and having a hollow center connecting first and second spaced apart ends, b) a plastic insertion tip integrally formed from or attached to said first end of said first member, said insertion tip having three or more adjacent petals, which petals have sides and wherein at least two of said petals (i) are joined by a membrane or (ii) have membranes extending from said sides narrowing any gap therebetween, and c) a second member sized for telescopic mounting in said second end of said first member, said second member adapted to expel said powder through said insertion tip as said second member is pushed through said hollow center of said first member. In some embodiments, each side of each petal has a membrane. In some embodiments the membrane(s) is pre-broken. In some embodiments, the insertion tip has five petals. In some embodiments, the powder applicator further comprises a cap.

In a further group of embodiments, the invention provides, a system for administering a powder to a vagina, said system comprising a powder applicator comprising a) a first member capable of housing a powder and having a hollow center connecting first and second spaced apart ends, b) a plastic insertion tip integrally formed from or attached to said first end of said first member, said insertion tip having three or more adjacent petals, which petals have sides and wherein at least two of said petals (i) are joined by a membrane or (ii) have membranes extending from said sides narrowing any gap therebetween, and c) a second member sized for telescopic mounting in said second end of said first member, said second member adapted to expel said powder through said insertion tip as said second member is pushed through the hollow center of said first member, and d) a powder. In some embodiments, the powder is of live bacteria. In some embodiments, the live bacteria are of the genus Lactobacillus. In some embodiments, the powder further comprises one or more excipients. In some embodiments the excipients comprise prebiotic oligosaccharides. Such prebiotic oligosaccharides might be selectively utilized by lactobacilli. In some embodiments, each of the petals has a membrane.

In a further group of embodiments, the invention provides methods of applying a powder to vaginal mucosa, the method comprising a) inserting into a vagina a powder applicator comprising i) a first member capable of housing a powder and having a hollow center connecting first and second spaced apart ends, ii) a plastic insertion tip integrally formed from or attached to said first end of said first member, said insertion tip having three or more adjacent petals, which petals have sides and wherein at least two of said petals (A) are joined by a membrane or (B) have membranes extending from said sides narrowing any gap therebetween, and iii) a second member sized for telescopic mounting in said second end of said first member, said second member adapted to expel said powder through said insertion tip as said second member is pushed through the hollow center of said first member, and b) pushing said second member through said hollow center of first member, thereby expelling said powder into said vagina, thereby applying said powder to said vaginal mucosa. In some embodiments, the powder is of live bacteria. In some embodiments, the live bacteria are of the genus Lactobacillus. In some embodiments, each side of each of said petals has a membrane.

In another embodiment, the invention provides applicators for applying powders to the vagina. The applicators comprise a) a first member capable of housing a powder having an outer and an inner surface, the inner surface forming the boundary of a hollow cavity with the hollow cavity connecting a first and a second spaced apart ends, b) a plastic insertion tip integrally formed from or said first end of said first member, said insertion tip having three or more adjacent petals, which petals have sides and wherein at least two of said petals (i) are joined by a membrane or (ii) have membranes extending from said sides narrowing any gap therebetween, wherein the first member has a gripping means and c) a second member sized for telescopic mounting in said second end of said first member, said second member adapted to expel said powder through said insertion tip as said second member is pushed through said hollow center of said first member wherein said first and second member has a latching means. In some embodiments, the gripping means is a spine on the outer surface of the first member. In some embodiments the latching means encompasses one or more rings. In some embodiments, latching means encompasses one or more rings and grooves.

### BRIEF DESCRIPTION OF THE DRAWINGS

FIG. 1 is an exploded view of a powder applicator.

FIG. 2 is a cross sectional view of a powder applicator loaded with powder.

FIG. 3 is an enlarged side view of an embodiment of an inserter tip.

FIG. 4 is an enlarged side view of an embodiment of an inserter tip having petals and membranes according to the present invention.

FIG. 5 is a cross sectional view of an embodiment of a powder applicator.

FIG. 6 is a perspective view of an embodiment of an outer tube.

FIG. 7(a) is a cross sectional view of an embodiment of an outer tube while FIG. 7(b) is a cross sectional view of an embodiment of a plunger.

FIG. 8(a) depicts an embodiment of an applicator before the powder is dispensed while FIG. 8(b) depicts the relative position of the plunger to the barrel after the powder is dispensed. Note that the Barrel is shown as it would be “undispensed.”

### DETAILED DESCRIPTION OF THE INVENTION

### Introduction

The present invention provides an improved applicator for delivering powders, such as dried or lyophilized bacteria or powdered botanical preparations, to the vaginal mucosa. Applicators designed to facilitate placement of catamenial tampons are, of course, well known in the art and are described in, for example, U.S. Pat. Nos. 5,928,183 and 6,890,324. In a common form, tampon applicators have a two piece telescopically assembled design. The tampon is housed in an outer tube or barrel and is expelled when desired by an inner member telescopically engaged or mounted within the outer tube and which acts as a plunger when it is advanced down the hollow center of the outer tube (for convenience of reference, the inner member is sometimes referred to herein as the “plunger”). The dimensions of tubes suitable for use in tampon applicators are well known in the art and can be used for applicators according to the present invention. Applicators designed for the delivery of gels and creams to the vagina, or for the placement of solid vaginal dosage forms such as tablets, capsules or suppositories, and the dimensions of such applicators are also well known in the art. Plastics suitable for use in such applicators are well known and can be used for applicators according to the present invention. In preferred embodiments, the applicator is formed from low density or linear low density polyethylene (LLDPE).

The front end (the end intended for insertion into the vagina) of some tampon applicators is open, leaving the tampon exposed, while others have a closed end to protect the tampon from debris or other contamination prior to use. Some applicators use a film membrane, or folds or pleats. Other applicators have a plurality of flexible “petals” at the front end. The petals flex outward when required to allow the tampon to be expelled. The petals are often formed into a rounded shape, such as a dome, which is perceived by the consumer as easier to insert than a blunt-ended applicator. The dome or other rounded shape may be referred to as an insertion tip.

In most cases, the petals are either pre-formed during the molding process or by cutting through the material forming the end. In applicators made from cardboard or paper-like materials, the petals are not formed by cuts through the material along the whole length defining the side of the petal, but rather by score lines or a series of perforations. The petals then break apart at the score lines or perforations when the tampon is advanced forward for insertion into the vagina. See, e.g., U.S. Pat. No. 6,886,443. For convenience of reference, petals defined by cuts along the entire length of the side of the petal will be referred to herein as “cut-formed petals” and those defined by perforations along the length of the side will be referred to collectively as “perforation-formed petals.” Petals defined by scoring along the sides will similarly be referred to as “scoring-formed petals” where reference to those is made.

Currently available applicators for delivering tampons, gels, or creams to the vagina are not suitable for the administration of powders. Pre-formed or cut-formed petals typically leave large spaces between the petals which would permit powder to escape. This not only reduces the amount of powder available for administration, but also reduces consumer acceptance since powder lost from the applicator may be visible on surfaces or the user's hands. Additionally, the spaces between the petals can permit the entry of moisture that can cause aggregation of the powder or otherwise adversely affect the powder. Applicators made from commonly used plastics are typically made by injection molding. Technical limitations in the fabrication of injection molds do not permit the petals to be made with tolerances tight enough to avoid substantial gaps that would allow powder leakage.

The present invention solves these problems. The applicators of the invention are made from injection molded plastic in which the mold is designed to extend a thin membrane of plastic from the petals both at the sides and at the tip where the petals come together. The membranes extending from the petals effectively seal the spaces that other would exist between the edges of adjacent petals. The membranes are frangible and break apart when pressure is applied by the plunger pressing the powder against the petals.

In some embodiments, the membranes are “pre-broken”, by pressing either the plunger or another clean, blunt-tipped object through the tip prior to loading the applicator with powder. When the plunger or other object is withdrawn within a short period of time, the shape of the petals and properties of the plastic causes them to reclose, bringing the edges of the broken membranes back together closely enough so that when powder is loaded, it is retained. Optionally, but preferably, a cap conforming to the shape of the tip is placed over the applicator tip prior to or after loading the tip with powder to hold the petals in place during filling and shipping. After the cap is in place, the petals will tend to retain their position even if the cap is then removed. Pre-breaking the membranes is desirable when the plastic used for the tip is made separately from the barrel of the outer tube and is of softer plastic, or when the applicator will be filled with a relatively large amount of powder, requiring more force to expel the contents, or when a smoother release of powder is preferred. Since unbroken membranes tend to result in a sudden release of the powder, some users may prefer a smoother powder release. Pre-broken membranes provide a smoother release that requires less force to dispense the powder and may therefore provide a better subjective experience for the user.

In an embodiment, the petals are 0.023 inches thick at the base and taper to 0.015 thick at the tip. Typically, the gaps between the petals (which exist whether or not membranes are present) are 0.010 inches wide. The membranes run along the sides and tips of the petals and typically have thicknesses between 0.003 and 0.005 inches. When unbroken, the membranes fill the gap between the petals. When the membranes are pre-broken and the petals are allowed to reclose, the edges of the now-broken membranes come back together and fill or substantially fill the gap between the petals sufficiently to retain all or most of the powder within the applicator. Thus, even with pre-broken membranes, the gap between the petals that would otherwise permit powder to escape is either narrowed or eliminated altogether.

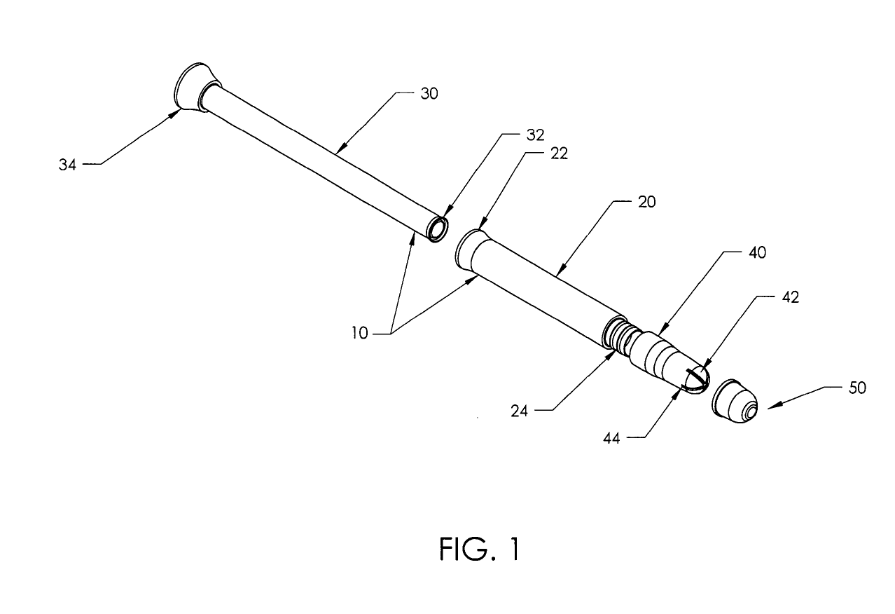

Referring to FIG. 1, a two piece applicator 10 is shown designed to permit introduction of powder into a woman's vagina. The applicator 10 includes a first member 20 and a second member 30. The first member 20, commonly referred to as an outer tube or barrel, preferably is a hollow tube which may later be filled with a desired quantity of powder to be administered. The tube is typically composed of materials strong enough not to collapse under pressure from vaginal walls and to have enough strength laterally to not collapse in the lateral direction when one end is inserted into a vagina. The first member 20 has an outer surface and an inner surface, with the inner surface forming the boundary of a cavity. The first member 20 has two ends, a first end, 22, to receive the second member 30 which can then be advanced into the cavity, and a second end, 24, which either is integrally formed into an insertion tip or, as in the embodiment shown in FIG. 1, attaching to a separately formed insertion tip, 40. The second member 30 likewise has two ends and acts as a plunger. Second member 30 is sized so that proximal end 32 can insert into the first member 20 through the first end 22 of the first member 20. The proximal end 32 is closed. The distal end 34 of the second member 30 may be open or closed, and may optionally widen out in diameter to provide a pressing means to facilitate pressing the second member 30 into the first member 20. The pressing means at the distal end 34 may be formed integrally as part of the second member 30, or provided by a separate piece 36 attached to the second member 30. The pressing means may also be in the form of a rounded recess sized to receive a finger, as described in U.S. Pat. No. 6,533,748.

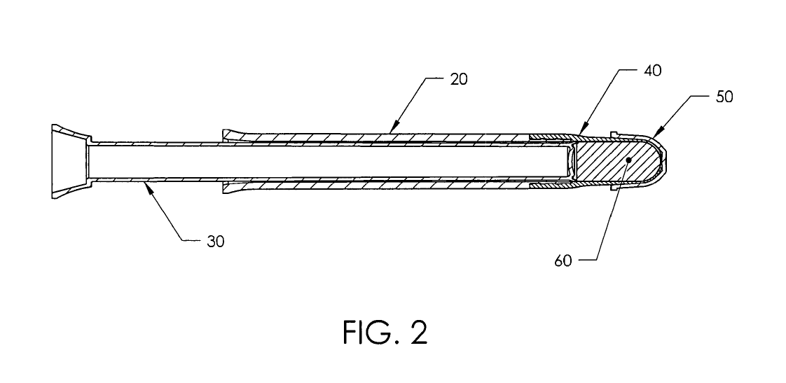

The insertion tip 40 has petals 42 defined by sides 44 in the material forming the insertion tip 40. Preferably, the insertion tip 40 is protected prior to use by a cap 50. FIG. 2 shows a cross-section view of applicator 10 loaded with powder 60. Second member 30 is inserted into first member, 20, and butts up against the powder 60 in the insertion tip 40, which is covered by a cap, 50.

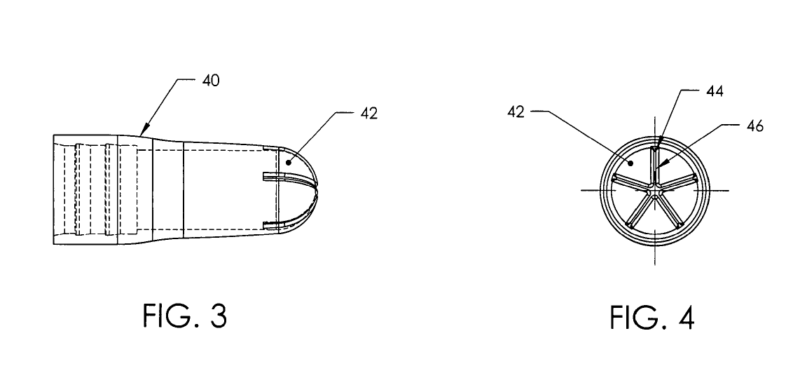

FIG. 3 shows a view of an embodiment of insertion tip 40. The insertion tip 40 is connected to first member 20 in a manner that permits pressure from the second member to force powder in the insertion tip 40 to be expelled. In preferred embodiments, the insertion tip 40 is flowably connected to first member 20.

Petals 42 are defined by sides 44 in the material. Along the sides 44 of the petals 42 are membranes 46, which are thinner than the body of the petals 42. If unbroken, the membranes eliminate any gap between the sides 44 of the petals 42. As described above, the membranes 46 are preferably “pre-cut” or “pre-broken” to reduce the pressure needed to expel powder from the applicator. Following separation of the membranes, the object forcing the membranes apart is withdrawn, permitting the petals 42 to reclose. The membranes 46 then serve to reduce or eliminate the gap that would otherwise exist between the petals 42.

FIG. 4 is a head-on view of insertion tip 40 more clearly showing the petals 42 and membranes 46. Preferably, the insertion tip 40 comprises three or more petals 42, with three to nine petals being preferred. In a particularly preferred embodiment, the insertion tip 40 has five petals 42.

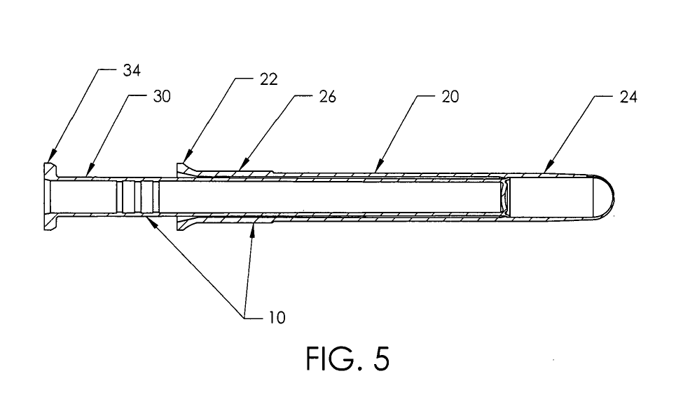

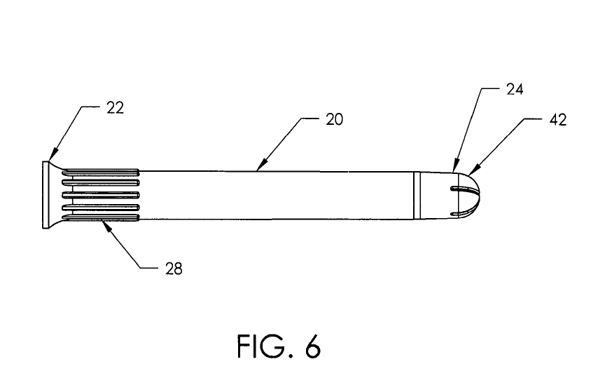

FIG. 5 shows an embodiment of the two piece applicator 10 with the second member 30 inserted into the first member 20. End 22 of first member 20 may be flared so as to facilitate holding the first member 20 while the second member 30 is pressed into first member 20. Optionally, a gripping means 26 is provided to facilitate holding the first member 20 while the second member 30 is pressed into first member 20. As depicted in FIG. 5, the gripping means 26 is achieved by increasing the thickness of the material proximal to End 22 of first member 20. In a further embodiment, as shown in FIG. 6, the gripping means 26 are splines 28 on the outer surface of the first member 20. The splines 28 may be formed integrally as part of the first member 20, or provided by a separate piece attached to the first member 20. Optionally, the splines have a matte finish to further improve the ability of the user to grip the first member 20.

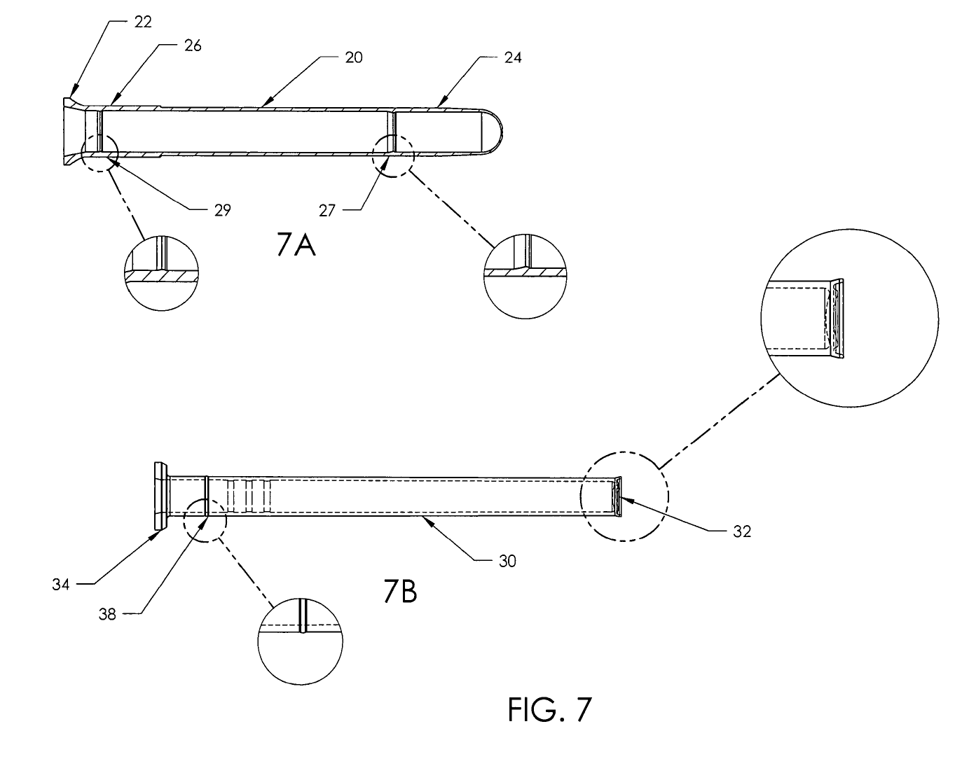

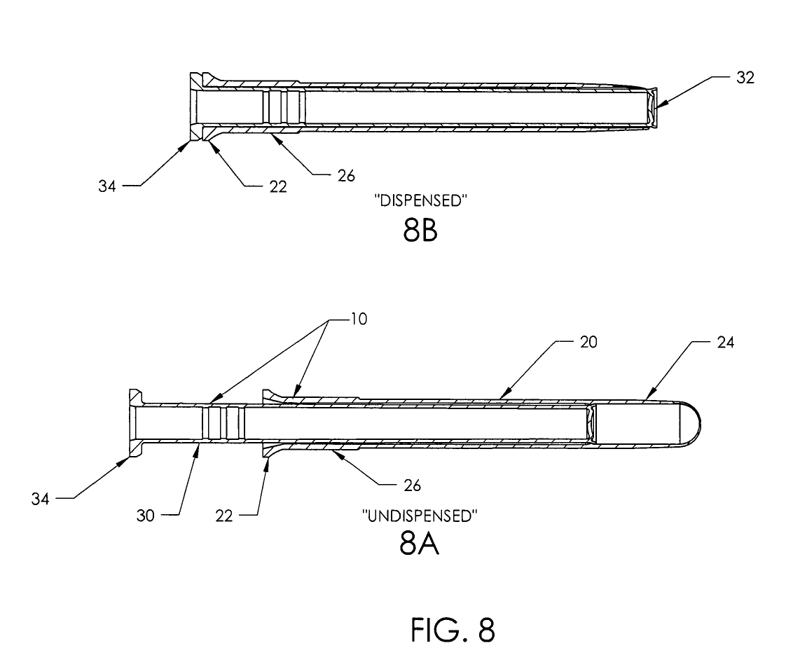

In another embodiment, a latching means is provided by two rings, 38 and 29, to impede the retraction of the second member 30, after powder expulsion. In a preferred embodiment, this latching means is composed of a ring 29, which is integral or attached to the inner surface of first member 20 and a second ring 38 which is integral or attached to the outer surface of second member 30, as shown in FIG. 7. In discharging the enclosed powder, ring 38 on the second member 30 traverses ring 29 on the first member 20. This latching means provides a friction point that acts as a locking mechanism thereby resisting return travel of the second member 30 through the first member 20. A second latching means is provided by ring 27 on the first member 20 and the proximal end 32 of the second member 30 that create a friction point to prevent accidental retraction of the second member 30 and spillage of powder through the distal end of the first member 20 prior to powder expulsion. In this embodiment, the first member 20 is filled with powder in an upright position and the second member 30 is inserted into the first member 20 such that the proximal end 32 of the second member 30 traverses ring 27 and is positioned between ring 27 and the top of the powder.

A representation of one embodiment is shown in FIG. 8 with the relative positions of the plunger and barrel as shown. Note the barrel is shown undispensed.

### Powders

The applicators are designed to facilitate the introduction of powder into the vagina. In an important group of embodiments, the powder is of a Lactobacillus that colonizes the vaginal mucosa. The Lactobacillus may be a natural species or strain thereof, or may be recombinantly altered to express biologically active proteins and uses thereof. In some embodiments, the powder comprises bacteria which are first prepared in a preservation matrix such as described in U.S. Pat. No. 6,468,526. Powders can also be made from bacteria in a dissolvable element made of dissolvable polymer material and/or complex carbohydrate material and/or proteinaceous material (e.g., gelatin) and/or other components (sugars, polyols, antioxidants, etc.) and/or other variations known to those skilled in the art, such that it remains in substantially solid form before use, and dissolves due to human body temperatures and moisture during use to release the agent material in a desired timed release and dosage. See, e.g., U.S. Pat. No. 5,529,782. The powder may also optionally include excipients or other agents to facilitate dispersion or dissolution of the powder, or to otherwise improve its characteristics, such as growth of the bacteria, for use in the vaginal environment.

While administration of powders of beneficial bacteria such as Lactobacillus are a preferred embodiment of the invention, applicators according to the present invention may be used to introduce into the vagina any agent deemed to be desirable, so long as it can be provided in powder form. For example, U.S. Pat. No. 6,888,043 describes a number of agents deemed to have beneficial or therapeutic properties that can be introduced into the vagina. These agents include botanical agents such as aloe vera, comfrey, calendula, tea extracts, lemon balm, as well as vitamins, minerals, and agents to treat infertility. Agents for which it is desirable to provide high local concentrations in the vaginal tract without having a high systemic concentration are particularly amenable for use in the applicators of the invention.

All terms herein not otherwise defined are intended to have the meaning normally accorded by persons of skill in the art. All publications, patents, and patent applications cited in this disclosure are herein incorporated by reference as if each individual publication, patent, or patent application were specifically and individually indicated to be incorporated by reference.

What is claimed is:

1. A powder applicator comprising
a) a first member capable of housing a powder and having a hollow center connecting first and second spaced apart ends,
b) a plastic insertion tip integrally formed from or attached to said first end of said first member, said insertion tip having three or more adjacent petals, which petals have sides and wherein at least two of said petals (i) are joined by a membrane or (ii) have membranes extending from said sides narrowing any gap therebetween, and
c) a second member sized for telescopic mounting in said second end of said first member, said second member adapted to expel said powder through said insertion tip as said second member is pushed through said hollow center of said first member.

2. The powder applicator of claim 1, wherein each side of each petal has a membrane.

3. The powder applicator of claim 1, wherein the membrane is pre-cut or pre-broken.

4. The powder applicator of claim 1, wherein said insertion tip has five petals.

5. The powder applicator of claim 1, further comprising a cap.

6. A system for administering a powder to a vagina, said system comprising a powder applicator comprising
a) a first member capable of housing a powder and having a hollow center connecting first and second spaced apart ends,
b) a plastic insertion tip integrally formed from or attached to said first end of said first member, said insertion tip having three or more adjacent petals, which petals have sides and wherein at least two of said petals (i) are joined by a membrane or (ii) have membranes extending from said sides narrowing any gap therebetween, and
c) a second member sized for telescopic mounting in said second end of said first member, said second member adapted to expel said powder through said insertion tip as said second member is pushed through said first member, and
d) a powder.

7. The system of claim 5, wherein said powder is of live bacteria.

8. The system of claim 6, wherein said live bacteria are of the genus Lactobacillus.

9. The system of claim 5, wherein each side of each of said petals has a membrane.

10. A method of applying a powder to vaginal mucosa, the method comprising
a) inserting into a vagina a powder applicator comprising
i) a first member capable of housing a powder and having a hollow center connecting first and second spaced apart ends,
ii) a plastic insertion tip integrally formed from or attached to said first end of said first member, said insertion tip having three or more adjacent petals, which petals have sides and wherein at least two of said petals (A) are joined by a membrane or (B) have membranes extending from said sides narrowing any gap therebetween, and
iii) a second member sized for telescopic mounting in said second end of said first member, said second member adapted to expel said powder through said insertion tip as said second member is pushed through said first member, and
b) pushing said second member through said first member, thereby expelling said powder into said vagina,
thereby applying said powder to said vaginal mucosa.

11. The method of claim 9, wherein said powder is of live bacteria.

12. The method of claim 10, wherein said live bacteria are of the genus Lactobacillus.

13. The system of claim 9, wherein each side of each of said petals has a membrane.

14. A powder applicator comprising
a) a first member capable of housing a powder having an outer and an inner surface, the inner surface forming the boundary of a hollow cavity with the hollow cavity connecting first and second spaced apart ends,
b) a plastic insertion tip integrally formed from said first end of said first member, said insertion tip having three or more adjacent petals, which petals have sides and wherein at least two of said petals (i) are joined by a membrane or (ii) have membranes extending from said sides narrowing any gap therebetween, wherein the first member has a gripping means; and
c) a second member sized for telescopic mounting in said second end of said first member, said second member adapted to expel said powder through said insertion tip as said second member is pushed through said hollow center of said first member, wherein said first member and said second member has a latching means.

15. The powder applicator of claim 13, wherein the gripping means is a spine on the outer surface of the first member.

16. The powder applicator of claim 13, wherein the latching means comprises one or more rings.

17. The powder applicator of claim 13, wherein the latching means comprises one or more rings and grooves.

18. The powder applicator of claim 13, wherein the membrane is pre-cut or pre-broken.

19. The powder applicator of claim 13, wherein the insertion tip has five petals.
